(19) United States
(12) Patent Application Publication
(10) Pub. No.: US 2022/0113126 A1
(43) Pub. Date: Apr. 14, 2022

(54) ASSESSMENT OF THE APPLIED HIDING OF A COATING

(71) Applicant: THE CHEMOURS COMPANY FC, LLC, WILMINGTON, DE (US)

(72) Inventor: STEVEN NICO DE BACKER, HAGEBEUK 10 (BE)

(73) Assignee: THE CHEMOURS COMPANY FC, LLC, WILMINGTON, DE (US)

(21) Appl. No.: 17/430,022

(22) PCT Filed: Feb. 18, 2020
(86) PCT No.: PCT/US2020/018586

§ 371 Date: Aug. 11, 2021

Related U.S. Application Data
(60) Provisional application No. 62/807,447, filed on Feb. 19, 2019.

(51) Int. Cl. G01B 11/06; G01N 21/84; G01N 21/55; G01N 33/32; G06T 7/00

(52) U.S. Cl. CPC G01B 11/0625; G01N 21/8422; G01N 2021/8427; G01N 33/32; G06T 7/0004; G01N 21/55

(57) ABSTRACT

A hiding value of a coating on a coated surface is assessed by assessing a reflectance of a multiplicity of pixels of an optically scanned image of the coated surface, and, based at least in part on the reflectance of each pixel of the multiplicity of pixels, assessing a hiding value of the coating on the coated surface.

### TECHNICAL FIELD

This invention relates to assessment of the applied hiding of a coating.

### BACKGROUND

The hiding of a coating can be used to determine the quality and effectiveness of the coating. For example, coatings with higher applied hiding demonstrate better perceived coverage and smoothness compared to coatings with lower applied hiding. Methods of determining the intrinsic hiding of coatings are known. However, testing methods used to determine intrinsic hiding require a uniform application of the relevant coating. While such an application can be realized under laboratory conditions, the precise, uniform application underlying the measurement of intrinsic hiding is often not realized in practice, such as when the paint is applied with a roller. As such, current methods of determining hiding are not effective for determining the applied hiding of a coating when the coating is applied in typical conditions outside the laboratory setting.

### SUMMARY

In a first general aspect, assessing a hiding value of a coating on a coated surface includes assessing a reflectance of a multiplicity of pixels of an optically scanned image of the coated surface, and, based at least in part on the reflectance of each pixel of the multiplicity of pixels, assessing a hiding value of the coating on the coated surface.

Implementations of the first general aspect may include one or more of the following features.

Assessing a hiding value of a coating on a coated surface may further include optically scanning the coated surface to yield the optically scanned image. In some cases, assessing the hiding value of the coating includes assessing a percentage of pixels having a reflectance of at least 70%. In some implementations, assessing the hiding value of the coating includes assessing a percentage of pixels having a reflectance of at least 80%.

Assessing a hiding value of a coating on a coated surface may further include assessing a thickness of the coating on the coated surface corresponding to each pixel of the multiplicity of pixels.

In a second general aspect, assessing a hiding value of a coating on a coated surface includes assigning a reflectance value to each pixel of a multiplicity of pixels of an optically scanned image of the coated surface, and assessing a percentage of the reflectance values at or above a threshold reflectance value.

Implementations of the second general aspect may include one or more of the following features.

In some cases, the optically scanned image is a grayscale image. In some implementations, assigning the reflectance value to each of the multiplicity of pixels includes converting a gray value associated with each pixel of the multiplicity of pixels to the reflectance value.

Assessing a hiding value of a coating on a coated surface may further include removing reflectance values outside a selected range from the reflectance values before assessing the percentage of the reflectance values at or above the threshold reflectance value. In some cases, the percentage of the reflectance values at or above the threshold reflectance value corresponds to a hiding value of the coating.

Assessing a hiding value of a coating on a coated surface may further include, based at least in part on the reflectance values, calculating a thickness of the coating corresponding to each pixel of the multiplicity of pixels.

In some cases, assessing a hiding value of a coating on a coated surface includes assessing a percentage of the reflectance values in one or more ranges of reflectance values less than the threshold reflectance value. Assessing a hiding value of a coating on a coated surface may further include graphically displaying the percentage of reflectance values in the one or more ranges of reflectance values and above the threshold value.

In a third general aspect, generating a hiding value of a coating on a coated surface includes generating, by one or more computer systems, a reflectance value of a multiplicity of pixels of an optically scanned image of the coated surface, generating, by one or more computer systems and based at least in part on the reflectance of each pixel of the multiplicity of pixels, a hiding value of the coating on the coated surface, and storing, on a hardware storage device, reflectance data representing the reflectance value in a first field of one or more data records and hiding data representing the hiding value in a second field of the one or more data records.

In a fourth general aspect, one or more machine-readable hardware storage devices store instructions that are executable by one or more processing devices to perform operations that include generating a reflectance value of a multiplicity of pixels of an optically scanned image of a coated surface, generating a hiding value of the coating on the coated surface based at least in part on the reflectance of each pixel of the multiplicity of pixels, and storing, on a hardware storage device, reflectance data representing the reflectance value in a first field of one or more data records and hiding data representing the hiding value in a second field of the one or more data records.

The details of one or more embodiments are set forth in the accompanying drawings and the description below. Other features, objects, and advantages will be apparent from the description, drawings, and claims.

### BRIEF DESCRIPTION OF DRAWINGS

FIG. 1 depicts a process for determining the applied hiding of a coating.

FIG. 2 shows a graphical display of the percentages of pixels of optically scanned images of coatings that fall within one or more reflectance ranges.

FIGS. 3A-3C show three exemplary architectural coating panels.

FIG. 4 shows the correlation between the reflectance values of pixels of optically scanned images of coatings and roller loading.

FIG. 5 shows the correlation between the percentage of pixels of optically scanned images of coatings with a reflectance below 75% and the gravimetric thickness of the coatings.

FIG. 6 shows the correlation between the percentage of pixels of optically scanned images of coatings with a reflectance below 75% and the wet film thickness of the coatings.

FIG. 7 shows the correlation between the intrinsic hiding of coatings and the percentage of pixels of optically scanned images of the coatings with a reflectance below 75% for coatings with a 50 micron wet film thickness.

FIG. 8 is a flow chart depicting operations in a process for determining the applied hiding of a coating.

FIG. 9 depicts an example computer system that can be used to execute implementations of the present disclosure.

### DETAILED DESCRIPTION

The hiding of a coating is a measure of the quality and effectiveness of a coating, such as paint. Intrinsic hiding, based intrinsic properties of a paint such as the amount of titanium dioxide present, the efficiency of titanium dioxide scattering, and the whiteness of the paint, allows quantification and comparison of the performance of different coatings and can be calculated using the Kubelka-Munk equations. Applied hiding is typically a function of intrinsic hiding and other factors, such as coating thickness. While intrinsic hiding can be measured in a laboratory, it is difficult, based on intrinsic hiding values alone, to determine the amount of coating required to achieve full applied hiding in typical application outside the laboratory (e.g., application of paint with a roller). This is due at least in part to the fact that testing methods currently used to determine the reflectance and intrinsic hiding of coatings assume a highly uniform and controlled application, which is typically not achieved under normal conditions. For example, applying paints with poor flow properties or levelling paint in the wet stage typically results in a surface characterized by pronounced peaks and valleys in the dry stage, and the valleys will cause a decrease in the perceived applied hiding.

Applied hiding can be described as a mathematical function linking the three parameters described in Equations 1-3, below (i.e., intrinsic hiding, thickness, and surface structure):

Applied hiding≈Intrinsic hiding (1)

Applied hiding≈Thickness (2)

Applied hiding≈1/surface structure (3)

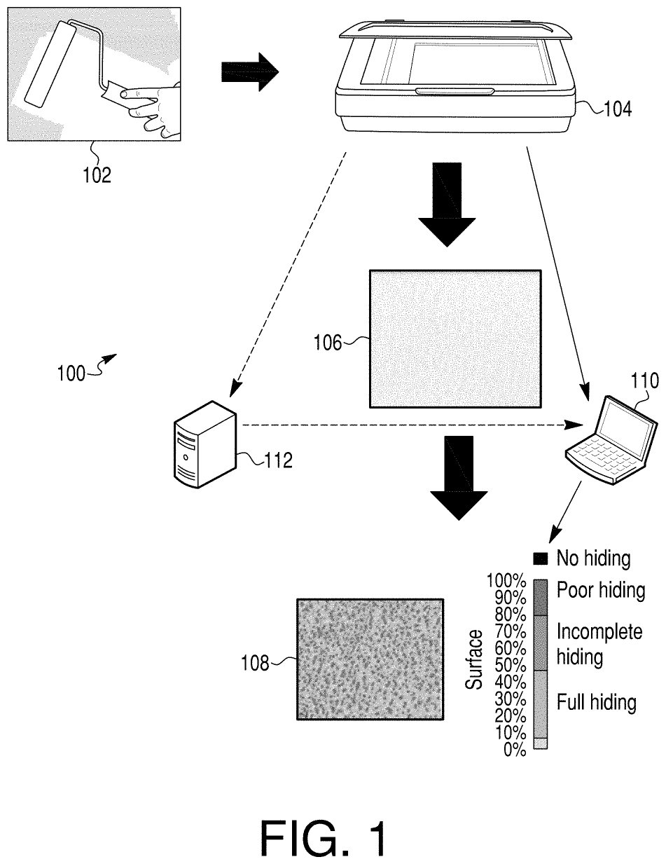

FIG. 1 depicts a process 100 for determining the applied hiding of a coating. In some implementations, a coated surface 102 is scanned with an optical scanner 104 to yield an optically scanned image 106. The optically scanned image 106 can be used to determine the reflectance of the coated surface 102.

In some implementations, the coated surface 102 is generated by applying a coat of paint to a surface. In one example, the coated surface 102 is generated by applying a white or light-colored paint to a black substrate. The white or light-colored paint typically has a tri-stimulus value of Y≥70, as provided in International Organization for Standardization (ISO) 6504, which is incorporated by reference herein. In some examples, the black substrate is a black chart manufactured by The Leneta Company. In some implementations, the coating is applied to the surface using a roller.

Coated surface 102 can be scanned using an optical scanner 104 to generate an optically scanned image 106 of the coated surface 102. In some implementations, the optical scanner 104 is a commercial flatbed scanner. In one example, the scanner 104 has a maximum optical resolution of 2400 dpi [dots per inch] along the charge-coupled device (CCD) sensor and a maximum mechanical resolution of 4800 dpi along the scan direction. Typically a scan-resolution can be selected from a list of available scan-resolutions (e.g., between 50 dpi up to 19200 dpi). In certain examples, a resolution of 240 dpi is suitable. This corresponds to 25400 [μm per inch]/240 [dots per inch]=105 μm per pixel.

In some implementations, the gray value for each pixel of the optically scanned image 106 of the coated surface 102 is assessed. As the scanner moves along the scanbed, the line-CCD captures the light, reflected by the material on the scanbed. The photons, impinging on any element of the line-CCD, create electrons in the CCD-element. Those electrons are “read out” and converted to a gray value by an analog-to-digital converter (ADC). To covert the signal levels to the grey level scale, a white reference material is scanned (in-built in the scanner) as well as the signal level with lamp off (black) is measured. These so-called “white points” and “black points” span the measureable gray value range of the scanner and are divided into a linear 8 bit or 16 bit scale by the ADC. The gray value is stored in the corresponding pixel in the digital image file.

In some implementations, a reflectance value (R) is generated for each pixel of the optically scanned image 106. In some examples, the reflectance value is generated for each pixel of the optically scanned image 106 based on the gray value measured for each pixel of the optically scanned image 106. For example, an empirical relationship between gray value and reflectance can be applied to the gray value measured for each pixel of the optically scanned image 106 to determine the reflectance of each pixel.

In some examples, the empirical relationship between gray value and reflectance is determined by (1) scanning multiple coated panels, each panel having a different gray level, to generate multiple optically scanned images; (2) measuring the gray level of each pixel of each optically scanned image; (3) calculating the reflectance of each pixel of each optically scanned image using the methods described in ISO 6504, and; (4) comparing the measured gray values to the calculated reflectance values.

In one example, as described here, the relationship R=f(G) can be used in converting from gray value G to reflection value to reflectance factor R. A set of reference panels, preferably made of the same material and appearance as the materials for which applied hiding is to be later assessed (e.g., paint). The set of reference materials are typically selected to cover a large range of reflectance values, preferably from R close to zero (“black”) up to R close to 1 (“white”). Panels can be scanned one by one, or patches from the panels can be organized as a step wedge or be represented as positions on a continuous variation of paint coating thickness. Preferably, the full range of reflectance values that are present in a sample to be analyzed is covered.

Preferably, the reference patches have uniform reflectance, at least within a region of the size of the measuring aperture (typically several mm) of a dedicated measurement device, e.g. a (spectro-)densitometer, which is used to record the reflectance values R. Uniform “bird coatings” are preferable to non-uniform “roll applications.”

The reflectance value R is measured with the measurement device for each patch or panel of the reference set. In some cases, the measurement spot locations of the measurement device are marked (e.g., by drawing a circle around the area), where the measuring aperture of the measurement device was located. That circle is typically somewhat greater in diameter than the measuring aperture. Such measurements may be carried out on various panel locations to verify the uniformity of R. The set of reference patches may also be scanned using specific TWAIN settings of a flatfield scanner. Then the average gray value G is measured inside the marked circle(s) (e.g., with image processing or image analysis tools).

The dataset of (G, R) for all measurement locations can be plotted in a XY-chart. A lookup table is constructed that interpolates between the measured points to cover the complete gray scale. In one example of interpolation, a mathematical function can be fitted to the dataset (e.g., using least-squares-fitting). In some cases, a polynomial of degree 3 may be used to fit the (G,R) data. This polynomial (f) is the mathematical expression of the relationship between G and R (“lookup-table”). For a polynomial of degree 3, it is characterized by 4 coefficients, which are input in the analytical results database (ARDB). The conversion from G in the scanned image to reflectance R is performed by applying this polynomial function f on the gray value G of every pixel in the scanned image, thus resulting in an image in which every pixel's value represents the reflectance value R. When scanners or scanner settings (e.g., brightness, contrast, gamma, or resolution) are changed, the process of determining R=f(G) can be repeated to yield f for the given parameters.

In some implementations, a matrix of reflectance values 108 corresponding to the reflectance value of each pixel of the optically scanned image 106 is generated on a local computing device 110, e.g., a desktop computer, a laptop computer, or a tablet computer. In some implementations, all or portions of process 100 can be performed on a remote computing device 112, e.g., a server system, e.g., a cloud-based server system. In some examples, one or more selected reflectance values are removed from the matrix. In one example, pixels that do not satisfy the definition of “white” per ISO 6504 are removed from the matrix of reflectance values 108.

Applied hiding can be determined for the coating 102 based on the reflectance values of each of the pixels of the optically scanned image 106. In some examples, the applied hiding of the coating 102 is assessed by determining the percentage of the pixels in the optically scanned image 106 having a reflectance value (R) above a threshold reflectance value. In some implementations, the threshold reflectance value is at least 0.7 or at least 0.8 (i.e., at least 70% or at least 80% reflectance). For example, the applied hiding of a coating 102 can be determined based on the percentage of pixels in an optically scanned image 106 of the coating 102 having a reflectance value above 0.7 or 0.8. In some implementations, pixels having a reflectance value above the threshold reflectance value demonstrate full hiding.

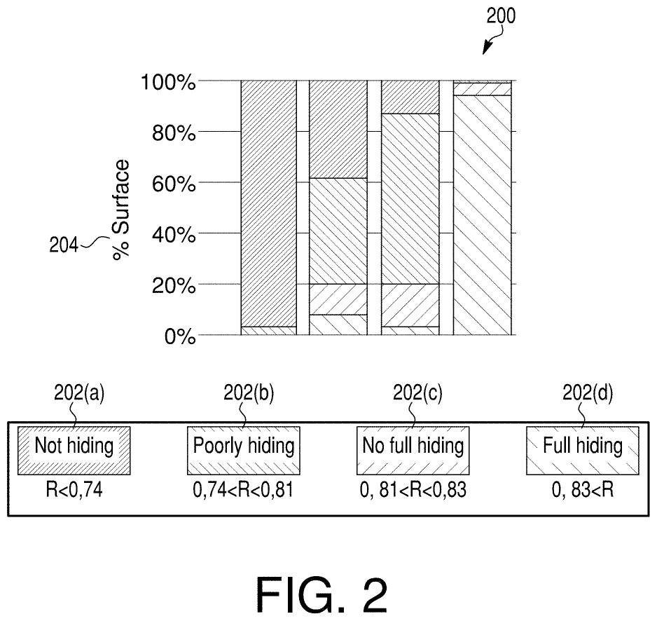

In some implementations, the percentage of pixels of an optically scanned image 106 of a coating 102 having a reflectance value in one or more ranges below the threshold value is assessed. In some examples, assessed reflectance value (R) ranges can include R≥0.8, 0.6≤R≤0.8, 0.4≤R≤0.6, and R≤0.4. In some implementations, the percentage of pixels of the optically scanned image 106 in each of the reflectance ranges can be graphically displayed. For example, as depicted in FIG. 2, a chart 200 may be generated that depicts the percentage of pixels 204 that fall within selected reflectance ranges 202 for coating 102.

In some implementations, the thickness of the coating corresponding to each pixel of the optically scanned image 106 of the coating 102 is determined based on the reflectance of each pixel. For example, using the Kubelka-Munk equations, such as Equations 4-6 below, the thickness of a coating 102 can be determined for each pixel of a scanned image 106 of the coating 102 based on the measured reflectance of each pixel and the applied hiding of the coating, as determined using the above-described method.

$$\begin{matrix}a = \frac{1}{2} [ R + \frac{{R}_{0} - R + {R}_{g}}{{R}_{0} {R}_{g}} ] & ( 4 ) \\ b = \sqrt{( {a}^{2} - 1 )} & ( 5 ) \\ S X = \frac{1}{b} Arc tgh [ \frac{1 - a {R}_{0}}{b {R}_{0}} ] & ( 6 )\end{matrix}$$

For Equations 4-6, above, R_{g} is the reflectance of the surface on which the coating 102 is applied, R is the reflectance of the coating 102 when applied to the surface, R₀ is the reflectance of the coating 102 when applied to an ideal black surface having an R_{g}=0, S is the coefficient of scatter, and X is the thickness of the coating 102. In some implementations, the coating 102 is homogenously applied under laboratory conditions, and the parameters R, R₀, R_{g}, and X for the homogenously applied coating 102 are measured using the techniques described in ISO Standard 6504. After determining the measured parameters R, R₀, R_{g}, and X for the homogenously applied coating 102, Equations 4-6 can then be used to calculate coefficients a, b, and S for the coating 102. After calculating coefficients a, b, and S, the same coating 102 can then be applied to a surface under typical conditions outside the laboratory setting (e.g., roller application), and the reflectance (R₀) of each pixel of an optically scanned image 106 of the coating 102 on the coated surface can be measured using the above-described scanner method. After determining R₀ for each pixel of the optically scanned image 106 of the coating 102, Equations 4-6 may be used in conjunction with the previously determined coefficients a, b, and S for the coating 102 to determine the thickness (X) of the coating 102 corresponding to each pixel. By combining the position of each pixel in the scanned image 106 with the thickness of the coating corresponding to each pixel, a 3D map of the surface of the applied coating 102 can be generated.

### Examples

A paint coating with a known intrinsic hiding value was applied to a substrate under controlled laboratory conditions at different thicknesses using a sagging applicator. The panel with the applied paint was scanned using a commercial scanner. The reflectance of each pixel of the scanned image was converted to the corresponding thickness using the Kubelka-Munk equations detailed in the American Society for Testing and Materials (ASTM) Standard D2805-11, which is incorporated by reference herein. As a control, the dry thickness of the paint coating was measured directly using a high resolution profilometer. A strong correlation was found between the thickness determined by application of the Kubelka-Munk equations to the reflectance values of the scanned image and the thickness measured by the profilometer. This strong correlation between thickness measurements demonstrates that the scanning techniques described above provide an accurate method for locally determining the thickness of a coating with a high resolution over a broad range of thicknesses.

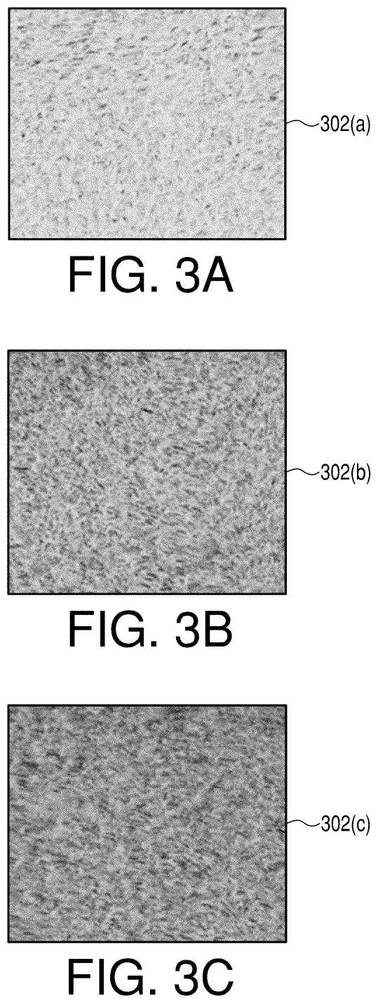

An architectural coating panel was created by applying paint with a saturated roller on a black chart. Without reloading of the roller, several other panels were generated to produce a series of panels in which each successive panel contained less paint. Each panel was weighed to determine the amount of paint on the panel. FIGS. 3A-3C show three exemplary panels 302 with paint applied by a roller. The roller loading applied to the panel 302(a) depicted in FIG. 3A was higher than the roller loading applied to the panels 302(b) and 302(c) depicted in FIGS. 3B and 3C, with panel 302(b) in FIG. 3B depicting intermediate roller loading and panel 302(c) in FIG. 3C depicting the lowest roller loading. As can be seen in FIGS. 3A-3C, increased roller loading resulted in better coverage of the black chart. However, regardless of the loading, each of the panels 302 included areas of chart that were not fully covered.

The panels 302 were scanned to generate scanned images of the coating, and the thicknesses of each panel coating and corresponding histograms were calculated based on the scanned images as described herein. Based on the scans, it was determined that the maximum thickness of the coatings increased with increased roller loading. The average gravimetric thickness (WFTG) of each panel coating was calculated based on the weight of each panel 302, the density (ρ) of the paint, and the covered surface area (A). The average gravimetric thickness for each panel was calculated following Equation 7, below:

WFTG=Weight/(ρA) (7)

The thicknesses calculated based on the scanner method described above and the thickness measures calculated based on the weight of the panels 302 using Equation 7 were highly correlated.

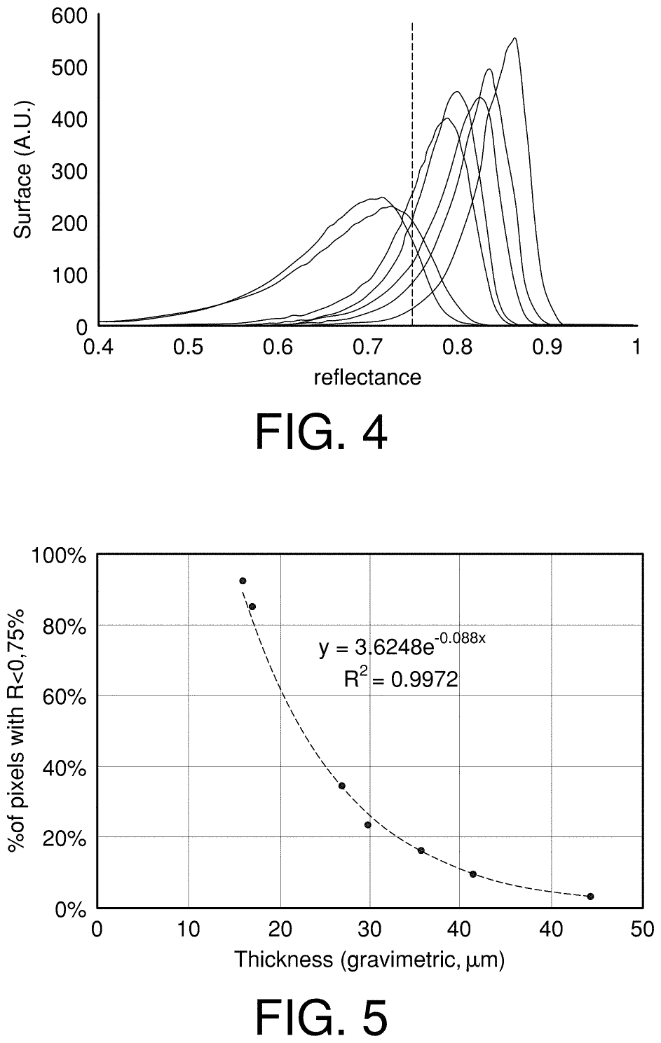

FIG. 4 shows the measured reflectance values for the scanned panel coatings. Areas of the coatings having a reflectance below 75% (R<75%) lowered the perceived applied hiding of the coatings. FIG. 5 shows the percentage of pixels of the optically scanned images of the panels 302 with a reflectance below 75% as a function of the gravimetric thickness of the paint coating (i.e., applied roller loading). As can be seen in FIG. 5, an exponential relationship exists between the percentage of pixels with a reflectance below 75% and the gravimetric thickness of the coating, indicating that increased roller loading results in higher reflectance, and, therefore, better applied hiding.

The test above was repeated with 11 commercial white paints, and a paint with Ti-Pure™ One Coat technology (developed by The Chemours Company), resulting in 80 panels. The commercial paints were selected to reflect different intrinsic hiding and rheologies. Consequently, the paints differed in composition and properties.

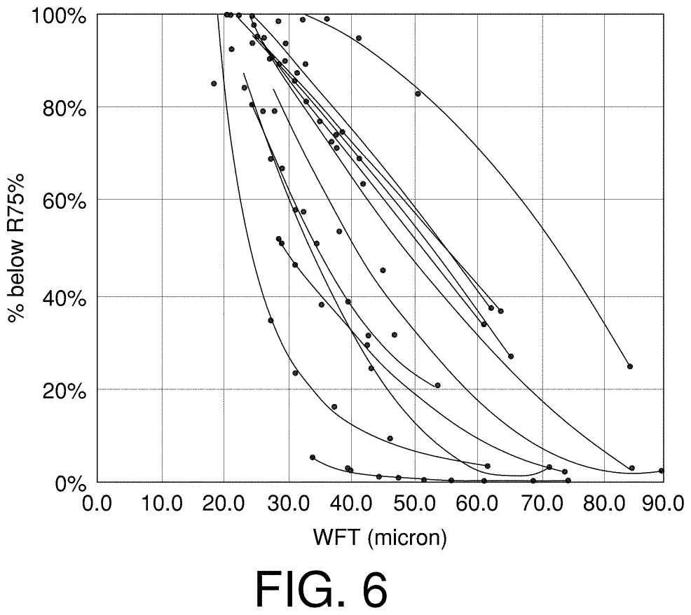

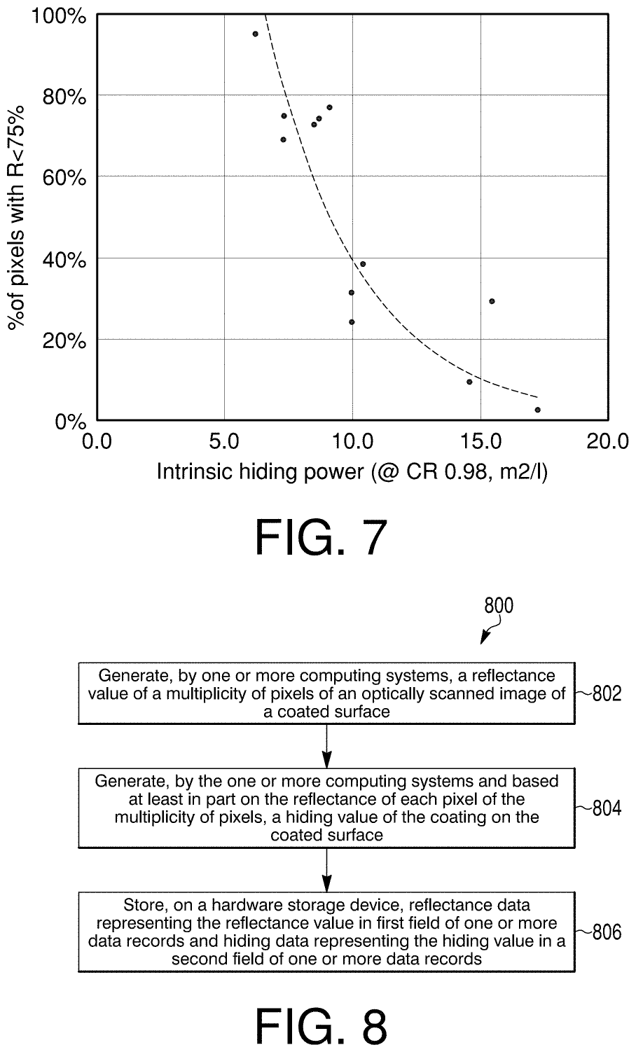

FIG. 6 shows the correlation between the percentage of pixels with a reflectance below 75% (R<75%) and the wet film thickness of the paint coating, which is a function of applied roller loading. The graph indicates that the percentage of the pixels with a reflectance below 75% decreases for all paints as the wet film thickness of the paint increases. However, the exact correlation between reflectance and wet film thickness varied strongly between each paint. As shown in FIG. 6, the largest variation between the different types of paints in the percentage of pixels with a reflectance below 75% was observed for samples with an approximately 50 micron wet film thickness. FIG. 7 shows the correlation observed between intrinsic hiding of the paint and a percentage of pixels with a reflectance below 75% for samples with a 50 micron wet film thickness. Fitting the curve in FIG. 7 to an exponential relation resulted in a correlation coefficient of about 0.78, indicating that 78% of the observed variation in percentages of pixels with a reflectance below 75% can be explained by the variation in intrinsic hiding between the different types of paint.

Visual observation of the 80 panels demonstrated that the surface structure of the dried paint films differed significantly between the different paints, as indicated by the presence of more or less pronounced peaks and valleys, where the valleys resulted in darker areas, causing lower perceived applied hiding.

The flow and leveling behavior of paint is generally understood to be a function of the paint's rheology profile. To better understand flow and levelling behavior, roughly equal weights of 3 architectural coatings of three different paints (paint A, paint B, and paint C) were applied to a chart using the same type of roller for each application. The compositions of the paints differed only in the rheology package. For example, paint A was highly Newtonian, paint C was highly pseudoplastic, and paint B was an intermediate case. The intrinsic hiding for each paint was the same.

The paints differed in flow behavior after application. Paint C was rated as having a poor flow, while paint A was rated as having the best flow, and paint B was given an intermediate rating. This observed flow behavior was in line with rheological characteristics of the different paints.

The black parts of the charts were scanned and the grey values were converted to three-dimensional (3D) map, as previously described. The 3D map for paint A showed an even surface, while the 3D map for paint C showed a more structured surface with an increased number of peaks and valleys.

The intrinsic flow (IF) behavior of paint was tested by minimizing the effects of roller type and other parameters linked with a manual roller application. The three paints (paint A, paint B, and paint C) were applied with a wired rod with a clearance of, for example 40 microns, on a black substrate manufactured by The Leneta Company. Paint C showed deep valleys and high mountains, indicating poor flow, while Paint A showed peaks that filled up the valleys, indicating good flow. This observation corresponds with the 3D images generated based on the roller applications of the same paints. A ratio of the average height of the surface peaks to the average depth of the surface valleys was provided as a quantitative measure of the flow behavior of the paint.

In another experiment, the previously tested 11 commercial white paints and the paint with Ti-Pure™ One Coat technology were applied with a 40 micron wired rod. The ratio of the surface peak height versus surface valley depth was calculated to serve as a measure of the intrinsic flow behavior and the surface structure of the applied paint.

The relationship between the applied hiding of a coating and the coating's wet film thickness (WFT), intrinsic hiding (IH), and intrinsic flow (IF) is described in Equation 5, below:

Ln(R<0.75)=0.097×IH+0.043 WFT+0.34 IF−0.0096×IH×WFT×IF (5)

Based on a regression analysis, it was determined that the applied hiding can be described by the following empirical equation (R²=0.96, R² pred=0.95). This equation allows for determination of the percentage of the surface of a paint coating having a reflectance below 75% (R<0.75%), which is demonstrated to be a good measure of the applied hiding of the coating. This percentage is determined based on the intrinsic hiding of the paint, the thickness at which the paint is applied, and the intrinsic flow of the paint. It can be assumed that the coefficients might change based upon the roller, the substrate, or the painter.

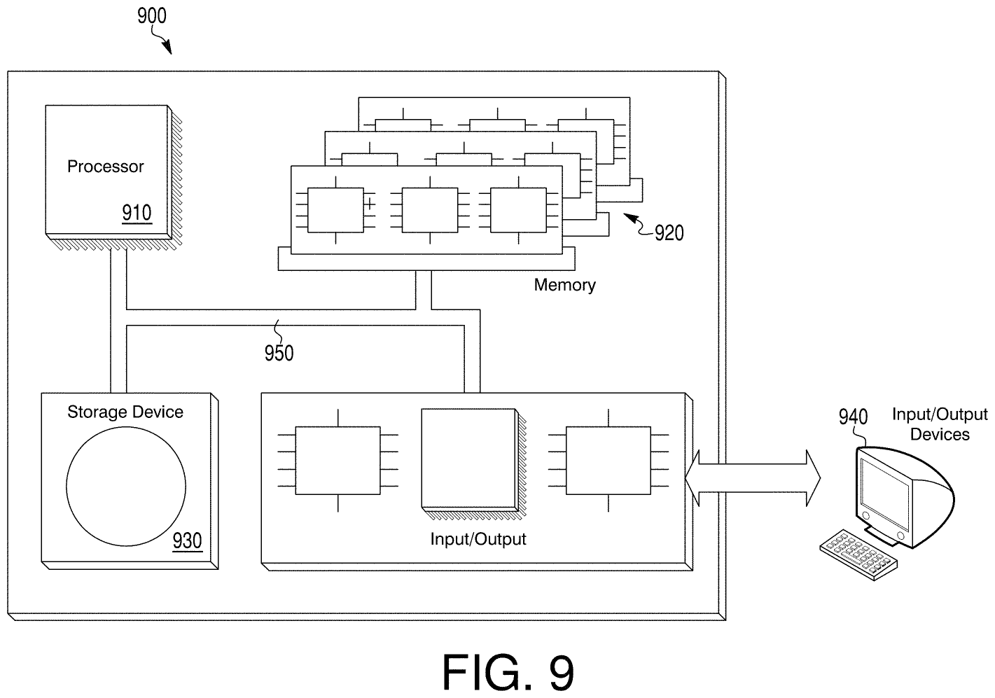

FIG. 8 depicts a flowchart of an example process for generating a hiding value of a coating on a coated surface. In some implementations, the process 800 can be provided as one or more computer-executable programs executed using one or more computing devices. In some examples, the process 800 is executed by a system such computer system 900 of FIG. 9, or a computing device. In some implementations, all or portions of process 800 can be performed on a local computing device, e.g., a desktop computer, a laptop computer, or a tablet computer. In some implementations, all or portions of process 800 can be performed on a remote computing device, e.g., a server system, e.g., a cloud-based server system.

A reflectance value of a multiplicity of pixels of an optically scanned image of a coated surface is generated by one or more computing systems (802). In some implementations, the optically scanned image is generated by using an optical scanner to scan a coated surface. In some implementations, the coated surface is generated by applying a coat of paint to a surface. In one example, the coated surface is generated by applying a white or light-colored paint to a black substrate. In some examples, the reflectance value is generated for each pixel of the optically scanned image based on the gray value measured for each pixel of the optically scanned image. For example, an empirical relationship between gray value and reflectance can be applied to the gray value measured for each pixel of the optically scanned image to determine the reflectance of each pixel. In one example, as previously described, the relationship R=f(G) can be used in converting from gray value G to reflection value to reflectance factor R.

A hiding value of the coating on the coated surface is generated by the one or more computing systems based at least in part on the reflectance of each pixel of the multiplicity of pixels (804). In some examples, the hiding value of the coating of the coated surface is assessed by determining the percentage of the pixels in the optically scanned image having a reflectance value (R) above a threshold reflectance value. In some implementations, the threshold reflectance value is at least 0.7 or at least 0.8 (i.e., at least 70% or at least 80% reflectance).

Reflectance data representing the reflectance value is stored on a hardware storage device in a first field of one or more data records and hiding data representing the hiding value is stored on the hardware storage device in a second field of one or more data records (806). In some implementations, the reflectance data is stored as a matrix.

FIG. 9 is a schematic diagram of a computer system 900. The system 900 can be used to carry out the operations described in association with any of the computer-implemented methods described previously, according to some implementations. For example, storage device 930 of system 900 can store instructions that are executable by one or more processing devices 910 to perform operations of generating a reflectance value of a multiplicity of pixels of an optically scanned image of the coated surface, generating a hiding value of the coating on the coated surface based at least in part on the reflectance of each pixel of the multiplicity of pixels, and storing, on hardware storage device 930, reflectance data representing the reflectance value in a first field of one or more data records and hiding data representing the hiding value in a second field of the one or more data records.

In some implementations, computing systems and devices and the functional operations described in this specification can be implemented in digital electronic circuitry, in tangibly-embodied computer software or firmware, in computer hardware, including the structures disclosed in this specification (e.g., system 900) and their structural equivalents, or in combinations of one or more of them. The system 900 is intended to include various forms of digital computers, such as laptops, desktops, workstations, personal digital assistants, servers, blade servers, mainframes, and other appropriate computers, including vehicles installed on base units or pod units of modular vehicles. The system 900 can also include mobile devices, such as personal digital assistants, cellular telephones, smartphones, and other similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 900 includes a processing device or processor 910, a memory 920, a storage device 930, and an input/output device 940. Each of the components 910, 920, 930, and 940 are interconnected using a system bus 950. The processor 910 is capable of processing instructions for execution within the system 900. The processor may be designed using any of a number of architectures. For example, the processor 910 may be a CISC (Complex Instruction Set Computers) processor, a RISC (Reduced Instruction Set Computer) processor, or a MISC (Minimal Instruction Set Computer) processor.

In one implementation, the processor 910 is a single-threaded processor. In another implementation, the processor 910 is a multi-threaded processor. The processor 910 is capable of processing instructions stored in the memory 920 or on the storage device 930 to display graphical information for a user interface on the input/output device 940.

The memory 920 stores information within the system 900. In one implementation, the memory 920 is a computer-readable medium. In one implementation, the memory 920 is a volatile memory unit. In another implementation, the memory 920 is a non-volatile memory unit.

The storage device 930 is capable of providing mass storage for the system 900. In some implementations, storage device 930 is a hardware-based storage device. In one implementation, the storage device 930 is a computer-readable medium. In various different implementations, the storage device 930 may be a floppy disk device, a hard disk device, an optical disk device, or a tape device.

The input/output device 940 provides input/output operations for the system 900. In one implementation, the input/output device 940 includes a keyboard and/or pointing device. In another implementation, the input/output device 940 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits). The machine learning model can run on Graphic Processing Units (GPUs) or custom machine learning inference accelerator hardware.

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A method of assessing a hiding value of a coating on a coated surface, the method comprising:
assessing a reflectance of a multiplicity of pixels of an optically scanned image of the coated surface; and
based at least in part on the reflectance of each pixel of the multiplicity of pixels, assessing a hiding value of the coating on the coated surface.

2. The method of claim 1, further comprising optically scanning the coated surface to yield the optically scanned image.

3. The method of claim 1, wherein assessing the hiding value of the coating comprises assessing a percentage of pixels having a reflectance of at least 70%.

4. The method of claim 3, wherein assessing the hiding value of the coating comprises assessing a percentage of pixels having a reflectance of at least 80%.

5. The method of claim 1, further comprising assessing a thickness of the coating on the coated surface corresponding to each pixel of the multiplicity of pixels.

6. A method of assessing a hiding value of a coating on a coated surface, the method comprising:
assigning a reflectance value to each pixel of a multiplicity of pixels of an optically scanned image of the coated surface; and
assessing a percentage of the reflectance values at or above a threshold reflectance value.

7. The method of claim 6, wherein the optically scanned image is a grayscale image.

8. The method of claim 7, wherein assigning the reflectance value to each of the multiplicity of pixels comprises converting a gray value associated with each pixel of the multiplicity of pixels to the reflectance value.

9. The method of claim 6, further comprising removing reflectance values outside a selected range from the reflectance values before assessing the percentage of the reflectance values at or above the threshold reflectance value.

10. The method of claim 6, wherein the percentage of the reflectance values at or above the threshold reflectance value corresponds to a hiding value of the coating.

11. The method of claim 6, further comprising, based at least in part on the reflectance values, calculating a thickness of the coating corresponding to each pixel of the multiplicity of pixels.

12. The method of claim 6, further comprising assessing a percentage of the reflectance values in one or more ranges of reflectance values less than the threshold reflectance value.

13. The method of claim 12, further comprising graphically displaying the percentage of reflectance values in the one or more ranges of reflectance values and above the threshold reflectance value.

14. A computer-implemented method of generating a hiding value of a coating on a coated surface, the method comprising:
generating by one or more computer systems a reflectance value of a multiplicity of pixels of an optically scanned image of the coated surface;
based at least in part on the reflectance of each pixel of the multiplicity of pixels, generating by one or more computer systems a hiding value of the coating on the coated surface; and
storing, on a hardware storage device, reflectance data representing the reflectance value in a first field of one or more data records and hiding data representing the hiding value in a second field of the one or more data records.

15. One or more machine-readable hardware storage devices storing instructions that are executable by one or more processing devices to perform operations comprising:
generating a reflectance value of a multiplicity of pixels of an optically scanned image of a coated surface;
based at least in part on the reflectance of each pixel of the multiplicity of pixels, generating a hiding value of a coating on the coated surface; and
storing, on a hardware storage device, reflectance data representing the reflectance value in a first field of one or more data records and hiding data representing the hiding value in a second field of the one or more data records.
